(19) United States
(12) Patent Application Publication
(10) Pub. No.: US 2015/0158829 A1
(43) Pub. Date: Jun. 11, 2015

(54) METHOD FOR PRODUCING AZIDE COMPOUND, AND METHOD FOR PRODUCING 1-H-TETRAZOLE DERIVATIVE

(71) Applicant: Nippon Soda Co., Ltd., Chiyoda-ku, Tokyo (JP)

(72) Inventor: Hidekazu Miyazaki, Takaoka-shi (JP)

(73) Assignee: Nippon Soda Co., Ltd., Chiyoda-ku, Tokyo (JP)

(21) Appl. No.: 14/406,602

(22) PCT Filed: Jun. 7, 2013
(86) PCT No.: PCT/JP2013/065833

§ 371 Date: Dec. 9, 2014

(30) Foreign Application Priority Data
Jun. 12, 2012 (JP) 2012-132812

(51) Int. Cl. C07D 257/04; C07C 247/04

(52) U.S. Cl. CPC C07D 257/04; C07C 247/04

(57) ABSTRACT

The present invention provides a method for producing an azide compound that includes reacting an alkylating agent or silylating agent with an azide represented by the following general formula (II) (wherein, M represents an alkaline metal atom or alkaline earth metal atom and m represents 1 or 2) in a flow reactor to produce an azide compound represented by the following general formula (I) (wherein, Y represents an alkyl group, arylalkyl group, substituted silyl group or substituted silylalkyl group) in the state of a solution.

M(N₃)ₘ (II)

Y—N₃ (I)

### TECHNICAL FIELD

The present invention relates to a method for producing an azide compound and a method for producing a 1H-tetrazole derivative. More particularly, the present invention relates to a method for synthesizing an azide compound by reacting in a flow reactor an azide of an alkaline metal or alkaline earth metal that has low risk of explosion and the like as raw material, and a method for producing a 1H-tetrazole derivative from the resulting azide compound.

The present application claims priority on the basis of Japanese Patent Application No. 2012-132812 filed in Japan on Jun. 12, 2012, the contents of which are incorporated herein by reference.

### BACKGROUND ART

Numerous types of control agents have been proposed for use against diseases of agricultural and horticultural crops. For example, Patent Document 1 discloses a tetrazoyl oxime derivative having superior pharmacological efficacy for useful plants, and proposes the use thereof as a plant disease control agent. Examples of methods for producing the tetrazoyl oxime derivative disclosed in Patent Document 1 include the method described in Patent Document 2 whereby a hydroxylamine is reacted with a 1-alkyl-5-benzoyl-1H-tetrazole derivative represented by the following general formula (P) and the resulting tetrazoylhydroxyimino derivative is used as raw material to produce a tetrazoyl oxime derivative. In general formula (P), A′ represents a halogen atom, alkyl group, alkoxy group, methanesulfonyl group, trifluoromethyl group, aryl group, cyano group or nitro group, n represents an integer of 0 to 5, and Y′ represents an optionally substituted alkyl group.

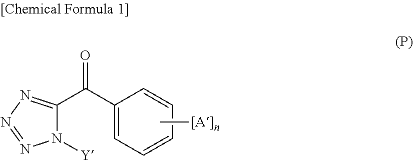

The method for producing the 1-alkyl-5-benzoyl-1H-tetrazole derivative represented by general formula (P) preferably consists of directly reacting benzoyl cyanide and an alkyl azide followed by forming a tetrazole ring by a cycloaddition reaction since it is easy to control the locations of substituents on the tetrazole ring.

An alkyl azide used as raw material of the aforementioned reaction is typically synthesized by alkylating sodium azide with an alkylating agent. For example, Non-Patent Document 1 reports that methyl azide was synthesized by methylating sodium azide with a methylating agent. Here, the alkyl azide is frequently a compound that is a highly explosive, highly dangerous compound in the manner of methyl azide and the like. Consequently, a method is sought that enables alkyl azide to be produced more safely and efficiently using comparatively safe sodium azide as raw material.

In addition, Patent Document 3 reports that a compound represented by general formula (P′) (wherein, A′ and n are the same as in general formula (P)) was able to be synthesized efficiently and safely by reacting an alkyl isocyanide such as methyl isocyanide with an acid halide such as benzoyl chloride followed by reacting the aforementioned reaction product with sodium azide. However, the method described in the aforementioned document requires that the methyl isocyanide be isolated due to concerns over explosion and toxicity. Moreover, it is extremely difficult to industrialize the aforementioned method due to the extremely distressing odor of methyl isocyanide.

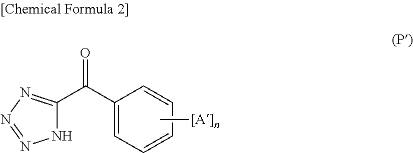

In addition, Non-Patent Document 2 reports that a tetrazole cyclization reaction was carried out by reacting benzyl cyanide and sodium azide in a flow reactor to a generate hydrogen azide a little at a time followed by immediately reacting with a nitrile. However, a reaction using benzoyl cyanide and methyl azide as raw materials is not disclosed in the aforementioned document.

### PRIOR ART DOCUMENTS

### Patent Documents

Patent Document 1: International Publication No. WO 2003/016303

Patent Document 2: International Publication No. WO 2010/103783

Patent Document 3: International Publication No. WO 2011/110651

### Non-Patent Documents

Non-Patent Document 1: Dimroth, O. and Wislicenus, W., Chemische Berichte, 1905, Vol. 38, p. 1573

Non-Patent Document 2: Gutmann, et al., Angewandte Chemie International Edition, 2010, Vol. 49(39), p. 7101-7105

### DISCLOSURE OF THE INVENTION

### Problems to be Solved by the Invention

An object of the present invention is to provide a method for efficiently and safely producing an azide compound in a flow reactor using a comparatively safe azide of an alkaline metal or alkaline earth metal as raw material, and a method for further producing a 1H-tetrazole derivative from the resulting azide compound.

### Means for Solving the Problems

As a result of conducting extensive studies to solve the aforementioned problems, the inventor of the present invention found that an azide compound can be industrially safely and efficiently produced using an azide of an alkaline metal or alkaline earth metal as raw material by using a flow reactor for the reaction vessel, thereby leading to completion of the present invention.

Namely, the method for producing an azide compound and the method for producing a 1H-tetrazole derivative of the present invention include the aspects described in [1] to [19] below.

[1] A method for producing an azide compound, including: reacting an alkylating agent or silylating agent with an azide represented by the following general formula (II) (wherein, M represents an alkaline metal atom or alkaline earth metal atom and m represents 1 or 2) in a flow reactor to produce an azide compound represented by the following general formula (I) (wherein, Y represents an alkyl group, arylalkyl group, substituted silyl group or substituted silylalkyl group) in the state of a solution.

[Chemical Formula 3]

M(N₃)ₘ (II)

Y—N₃ (I)

[2] The method for producing an azide compound described in [1] above, wherein the alkylating agent or the silylating agent is a compound represented by any of the following general formulas (III-A) to (III-E) (wherein, Y is the same as defined in the general formula (I) and X represents a halogen atom).

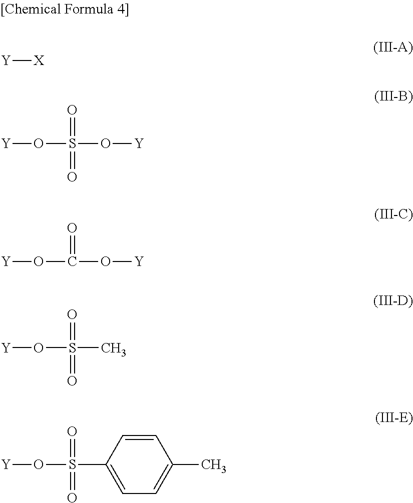

[3] The method for producing an azide compound described in [1] or [2] above, wherein a solution containing the alkylating agent or the silylating agent and a solution containing the azide represented by the general formula (II) are respectively and separately introduced into the flow reactor.

[4] The method for producing an azide compound described in [3] above, wherein the solvent of the solution containing the azide represented by the general formula (II) contains water, and the solvent of the solution containing the alkylating agent or the silylating agent is an organic solvent capable of dissolving the azide compound represented by the general formula (I) and capable of undergoing phase separation with water.

[5] The method for producing an azide compound described in [4] above, wherein the organic solvent is toluene.

[6] The method for producing an azide compound described in any of [3] to [5] above, wherein the solution containing the azide represented by the general formula (II) further contains a base.

[7] The method for producing an azide compound described in any of [1] to [6] above, wherein Y represents an alkyl group.

[8] A method for producing a 1H-tetrazole derivative, including: reacting an alkylating agent or a silylating agent with an azide represented by the following general formula (II) (wherein, M represents an alkaline metal atom or alkaline earth metal atom, and m represents 1 or 2) in a solution in a first reactor portion of a flow reactor to produce an azide compound represented by the following general formula (I) (wherein, Y represents an alkyl group, arylalkyl group, substituted silyl group or substituted silylalkyl group), followed by mixing the solution containing the azide compound with a solution containing a cyanide compound represented by the following general formula (IV) (wherein, Z represents —CO—, —SO₂— or —CRₐR_{b}— (wherein, Rₐ and R_{b} respectively and independently represent a hydrogen atom, alkyl group or unsubstituted or substituted aryl group), p represents 0 or 1, q represents 0 or 1, r represents 0 or 1, R¹ represents an alkyl group or hydrogen atom in the case q is 0 or an alkylene group in the case q is 1 and R² represents an unsubstituted or substituted aryl group, provided that q is 1 in the case p is 0 and q is 1 in the case r is 0) and reacting in a second mixer portion of the flow reactor to produce a compound represented by the following general formula (V) (wherein, Y, Z, R¹, R², p, q and r are the same as previously defined).

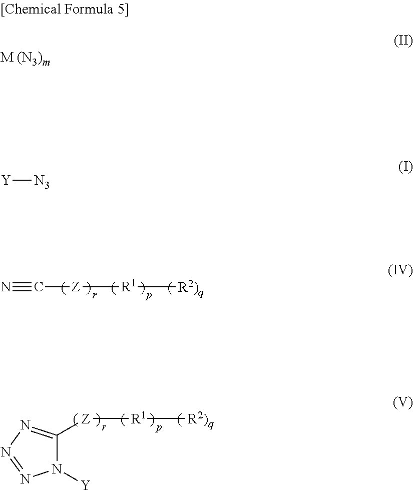

[9] The method for producing a 1H-tetrazole derivative described in [8] above, wherein the alkylating agent or the silylating agent is a compound represented by any of the general formulas (III-A) to (III-E) (wherein, Y is the same as defined in the general formula (I) and X represents a halogen atom).

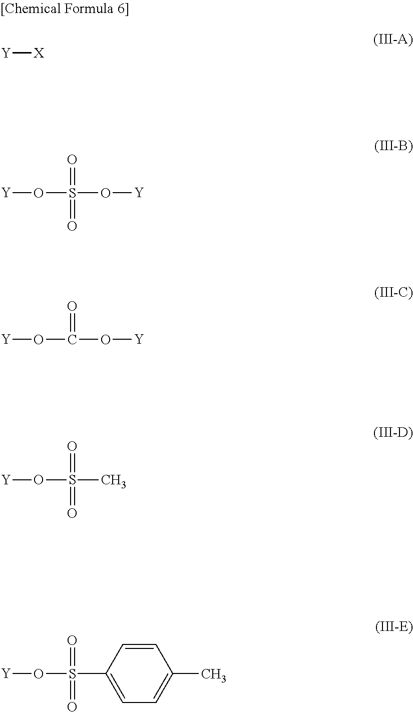

[10] The method for producing an azide compound described in [8] or [9] above, wherein a solution containing the alkylating agent or the silylating agent and a solution containing the azide represented by the general formula (II) are respectively and separately introduced into the flow reactor, both solutions are mixed in the first mixer portion, and the resulting mixture is introduced into the first reactor portion.

[11] The method for producing a 1H-tetrazole derivative described in [10], wherein the solvent of the solution containing the azide represented by the general formula (II) contains water, and the solvent of the solution containing the alkylating agent or the silylating agent is an organic solvent capable of dissolving the azide compound represented by the general formula (I) and capable of undergoing phase separation with water.

[12] The method for producing a 1H-tetrazole derivative described in [11] above, wherein the organic solvent is toluene.

[13] The method for producing a 1H-tetrazole derivative described in [11] or [12] above, wherein the solution containing the azide compound represented by the general formula (I) discharged from the first reactor portion is separated into an aqueous layer and an organic solvent layer, and the organic solvent layer is recovered and mixed with a solution containing the cyanide compound represented by the general formula (IV) in the second mixer portion.

[14] The method for producing an azide compound described in any of [10] to [13] above, wherein the solution containing the azide represented by the general formula (II) further contains a base.

[15] The method for producing a 1H-tetrazole derivative described in any of [8] to [14] above, wherein Y represents an alkyl group.

[16] The method for producing a 1H-tetrazole derivative described in any of [8] to [15] above, wherein R² is a group represented by the following general formula (s1) (wherein, A represents a halogen atom, alkyl group, haloalkyl group, alkoxy group, haloalkoxy group, alkylsulfonyl group, unsubstituted or substituted aryl group, cyano group or nitro group, n represents an integer of 0 to 5, A may be the same or different when n is 2 or more, and the asterisk (*) represents bonding to R¹ in the case p is 1 in the general formula (IV), bonding to Z in the case p is 0 and r is 1, or binding to a carbon atom of a cyanido group in the case p and r are 0).

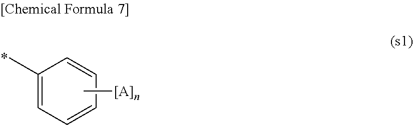

[17] The method for producing a 1H-tetrazole derivative described in any of [8] to [16] above, wherein the reaction temperature of the reaction between the azide compound represented by the general formula (I) and the cyanide compound represented by the general formula (IV) is 150° C. to 250° C.

[18] The method for producing a 1H-tetrazole derivative described in any of [8] to [17] above, wherein the solution containing the azide compound represented by the general formula (I) that is mixed with the solution containing the cyanide compound represented by the general formula (IV) contains the azide compound represented by the general formula (I) in an amount equal to 1.4 times or more the number of moles of the cyanide compound represented by general formula (IV).

[19] The method for producing a 1H-tetrazole derivative described in any of [8] to [18] above, wherein the solvent of the solutions mixed in the second mixer portion is toluene or N-methylpyrrolidone.

### Effects of the Invention

The method for producing an azide compound of the present invention enables an azide compound to be produced industrially safely in the state of a solution (namely, in the state of being dissolved in a solvent) by using a flow reactor.

In addition, the method for producing a 1H-tetrazole derivative of the present invention enables a 1H-tetrazole derivative to be produced by directly reacting an azide compound produced according to the method for producing an azide compound of the present invention with a cyanide compound without having to remove outside the flow reactor.

Namely, the method for producing a 1H-tetrazole derivative of the present invention is characterized by carrying out a stepwise reaction in a flow reactor using an azide of an alkaline metal or alkaline earth metal as raw material that has low risk of explosion or other danger. As a result, a 1H-tetrazole derivative, which has substituents at the 1-position and 5-position and is useful as a synthesis raw material of active ingredients of agricultural chemicals and various other types of chemicals, can be produced industrially safely, in a short period of time and with high efficiency.

### BEST MODE FOR CARRYING OUT THE INVENTION

Although the following provides an explanation of preferred embodiments of the present invention, the present invention is not limited to these embodiments. Constituents of the present invention can be added, omitted, substituted or altered in other ways within a range that does not deviate from the gist of the present invention.

<Method for Producing Azide Compound>

The method for producing an azide compound of the present invention is characterized by reacting an alkylating agent or a silylating agent with an azide represented by the following general formula (II) in a flow reactor to produce an azide compound represented by the following general formula (I) in the state of a solution. In other words, the azide compound produced according to the method for producing an azide compound of the present invention can be recovered as a solution.

[Chemical Formula 8]

M(N₃)ₘ (II)

(In general formula (II), M represents an alkaline metal atom or alkaline earth metal atom and m represents 1 or 2.)

[Chemical Formula 9]

Y—N₃ (I)

(In general formula (I) Y represents an alkyl group, arylalkyl group, substituted silyl group or substituted silylalkyl group.)

There are some azide compounds in the manner of methyl azide that have a boiling point that is lower than the reaction temperature between an azide and alkylating agent and the like. Therefore, azide compounds in the gaseous state are removed from the reaction system by a procedure such as distillation. As a result, since the azide compound is recovered in the state of liquid having an extremely high concentration, there is a high risk of explosion and the like. In contrast, in the method for producing an azide compound of the present invention, since a flow reactor is used for the reaction vessel, the reaction can be carried out at a suitable pressure and the synthesized azide compound can be recovered in a state of being dissolved in a solvent.

[Alkylating Agent or Silylating Agent]

In the method for producing an azide compound of the present invention, the sodium ion of the azide represented by general formula (II) is substituted with an alkyl group, arylalkyl group, substituted silyl group or substituted silylalkyl group by an alkylating agent or silylating agent. As a result, the azide compound represented by general formula (I) can be synthesized efficiently.

There are no particular limitations on the alkylating agent or silylating agent used in the present invention provided it is a compound that is able to substitute a sodium ion of the azide represented by general formula (II) with an alkyl group, arylalkyl group, substituted silyl group or substituted silylalkyl group, and can be suitably selected from among known alkylating agents and silylating agents.

The alkylating agent or silylating agent used in the present invention (to also be simply referred to as a “substituent Y introducing agent”) is preferably a compound represented by any of the following general formulas (III-A) to (III-E). Furthermore, these compounds can be synthesized from known compounds using known chemical reactions.

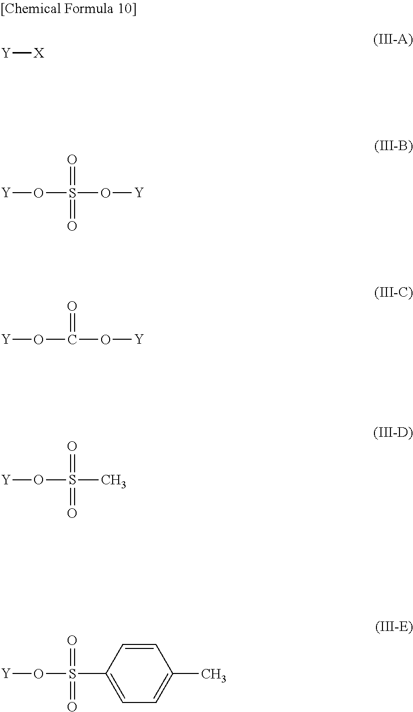

(In general formulas (III-A) to (III-E), Y is the same as defined in general formula (I) and X represents a halogen atom.)

In general formulas (III-A) to (III-E), Y represents an alkyl group, unsubstituted or substituted arylalkyl group, substituted silyl group or substituted silylalkyl group.

An alkyl group may be a linear alkyl group, branched alkyl group or cyclic alkyl group. The aforementioned alkyl group is preferably a linear alkyl group having 1 to 8 carbon atoms, a branched alkyl group having 3 to 8 carbon atoms or a cyclic alkyl group having 3 to 8 carbon atoms. Specific examples include a methyl group, ethyl group, n-propyl group, i-propyl group, n-butyl group, i-butyl group, s-butyl group, t-butyl group, n-pentyl group, n-hexyl group, cyclopropyl group, cyclobutyl group, cyclopentyl group and cyclohexyl group.

An unsubstituted or substituted arylalkyl group refers to a group in which at least one hydrogen atom of the alkyl group is substituted with an unsubstituted or substituted aryl group. The alkyl group substituted with an aryl group may be a linear alkyl group, branched alkyl group or cyclic alkyl group. The aforementioned alkyl group is preferably a linear alkyl group having 1 to 8 carbon atoms, a branched alkyl group having 3 to 8 carbon atoms or a cyclic alkyl group having 3 to 8 carbon atoms, and more preferably a linear alkyl group having 1 to 8 carbon atoms. In addition, the aryl group that becomes a substituent of the alkyl group may be a monocyclic group or polycyclic group. In the case of a polycyclic aryl group, as long as at least one of the rings is an aromatic ring, the remaining rings may be saturated rings, unsaturated rings or aromatic rings. In addition, in the case the aforementioned aryl group has a substituent, there are no particular limitations on the aforementioned substituent provided it is a chemically acceptable substituent, and specific examples thereof include the substituents listed in (1) to (85) to be subsequently described. Y in general formulas (III-A) to (III-E) is preferably a group in which one hydrogen atom of a linear alkyl group having 1 to 8 carbon atoms is substituted with an unsubstituted or substituted aryl group having 6 to 10 carbon atoms, more preferably a group in which one hydrogen atom of a linear alkyl group having 1 to 8 carbon atoms is substituted with an unsubstituted or substituted phenyl group, and even more preferably a group in which one hydrogen atom of a linear alkyl group having 1 to 8 carbon atoms is substituted with an unsubstituted phenyl group, and still more preferably a benzyl group.

A substituted silyl group is a group in which at least one hydrogen atom of the silyl group is substituted. There are no particular limitations on the substituent provided it is a chemically acceptable substituent. Specific examples thereof include the substituents listed in (1) to (85) to be subsequently described. In addition, in the case 2 or 3 hydrogen atoms are substituted, the substituents may be mutually the same or different. Y in general formulas (III-A) to (III-E) is preferably a silyl group in which 1 to 3 hydrogen atoms are substituted with the same or different alkyl groups, more preferably a silyl group in which three hydrogen atoms are substituted with the same or different alkyl groups, even more preferably a trimethylsilyl group, triethylsilyl group, ethyldimethylsilyl group or t-butyldimethylsilyl group, and still more preferably a trimethylsilyl group.

A substituted silylalkyl group is a group in which at least one hydrogen atom of the alkyl group is substituted with a substituted silyl group. The alkyl group substituted with the silyl group may be a linear alkyl group, branched alkyl group or cyclic alkyl group. The aforementioned alkyl group is preferably a linear alkyl group having 1 to 8 carbon atoms, a branched alkyl group having 3 to 8 carbon atoms or a cyclic alkyl group having 3 to 8 carbon atoms, and is more preferably a linear alkyl group having 1 to 8 carbon atoms. In addition, examples of silyl groups serving as a substituent of the alkyl group are the same as in the examples of the aforementioned substituted silyl group. Y in general formulas (III-A) to (III-E) is preferably a group in which one hydrogen atom of a linear alkyl group having 1 to 8 carbon atoms is substituted with a substituted silyl group, more preferably a group in which one hydrogen atom of a linear alkyl group having 1 to 8 carbon atoms is substituted with a silyl group in which one to three hydrogen atoms are substituted with the same or different alkyl groups, even more preferably a group in which one hydrogen atom of a linear alkyl group having 1 to 3 carbon atoms is substituted with a trimethylsilyl group, triethylsilyl group, ethyldimethylsilyl group or t-butyldimethylsilyl group, and still more preferably a trimethylsilylmethyl group.

The compound represented by general formulas (III-A) to (III-E) is preferably that in which Y represents a linear alkyl group having 1 to 8 carbon atoms, a branched alkyl group having 3 to 8 carbon atoms, a cyclic alkyl group having 3 to 8 carbon atoms or an unsubstituted or substituted benzyl group, more preferably that in which Y represents a linear alkyl group having 1 to 6 carbon atoms, a branched alkyl group having 3 to 6 carbon atoms or a cyclic alkyl group having 3 to 6 carbon atoms, and even more preferably that in which Y represents a linear alkyl group having 1 to 3 carbon atoms. Among these, the compound represented by general formulas (III-A) to (III-E) is particularly preferably that in which Y represents a methyl group, namely a halomethyl, dimethyl sulfate, dimethyl carbonate, methyl mesylate or methyl tosylate. In the method for producing an azide compound of the present invention, since the reaction is carried out in a flow reactor, an azide compound can be safely and efficiently produced in the state of a solution even in the case the synthesized product is a lower alkyl azide having 6 or fewer carbon atoms that is generally considered to be explosive and toxic due to the low boiling point thereof.

Although the substituent Y introducing agent used in the method for producing an azide compound of the present invention may be any of a compound represented by general formula (III-A) (halide), compound represented by general formula (III-B) (sulfate), compound represented by general formula (III-C) (carbonate), compound represented by general formula (III-D) (mesylate) or compound represented by general formula (III-E) (tosylate), it is preferably a compound represented by general formula (III-A) or a compound represented by general formula (III-B).

[Azide Represented by General Formula (II)]

In general formula (II), M represents an alkaline metal atom or alkaline earth metal atom. Examples of alkaline metal atoms include sodium, potassium and lithium, while examples of alkaline earth metal atoms include magnesium and calcium. Among these, M is preferably sodium or potassium and more preferably sodium.

In general formula (II), m represents an integer of 1 or 2. m represents 1 in the case M represents an alkaline metal atom, or m represents 2 in the case M represents an alkaline earth metal atom.

[Flow Reactor]

In the method for producing an azide compound of the present invention, a flow reactor is used for the reaction vessel used to react the azide represented by general formula (II) (to also be simply referred to as an “azide”) and the substituent Y introducing agent. Consequently, the reaction can be carried out comparatively safely in a low temperature environment even in a reaction in which a highly decomposable compound in the manner of an azide compound is obtained. Moreover, the synthesized azide compound can be recovered in the state of a solution by carrying out the reaction at a pressure of atmospheric pressure to 10 MPa.

The flow reactor used in the method for producing an azide compound of the present invention is provided with raw material introduction ports, a product discharge port and a flow path communicating there between. Raw materials are supplied through the aforementioned raw material introduction ports and the product obtained by a reaction that occurs in the aforementioned flow path is removed from the aforementioned product discharge port. The aforementioned flow path may also be provided with at least one of an introduction path, mixer portion and reactor portion (retention portion) as necessary. In the case the flow reactor is provided with a mixer portion, the portion of the flow path communicating between the raw material introduction ports and the mixer portion is referred to as an introduction path, while in the case a mixer portion is not present, the portion of the flow path that communicates between the raw material introduction ports and the reactor portion is referred to as an introduction path. In addition, the raw material introduction ports are normally connected to containers filled with raw materials. The product discharge portion may also be connected to a container for storing the product as necessary.

The mixer portion is a site that has the function of mixing a plurality of liquids by dispersion, and solutions supplied from the plurality of raw material introduction ports merge in the mixer portion. In addition, the reactor portion is a site where a reaction is carried out for synthesizing a product (azide compound represented by general formula (I) in the method for producing an azide compound of the present invention) from a plurality of raw material compounds (azide and substituent Y introducing agent in the method for producing an azide compound of the present invention). In the case of being provided with both a mixer portion and a reactor portion, the mixer portion is provided on the side of the raw material introduction ports. In the case of preliminarily supplying a reaction solution obtained by mixing all of the raw material compounds from a single raw material introduction port, the mixer portion need not be provided. In addition, the mixer portion may also serve as a reactor portion and the reactor portion need not be provided in the case the time required by the reaction for synthesizing the product is short and the reaction is able to be completed by the time the raw materials pass through the mixer portion.

In the case the flow reactor is provided with a plurality of raw material introduction ports and introduction paths, the upstream side of the flow path of the aforementioned flow reactor has a configuration in which it is branched corresponding to the number of introduction paths, and is further provided with at least one mixer portion. There are no particular limitations on the number of raw material introduction ports and introduction paths, and can be suitably selected corresponding the particular purpose. In the case of having three or more raw material introduction ports and introduction paths, a configuration may be employed in which liquid supplied from all of the introduction ports merges in a single mixer portion, or a configuration may be employed in which the liquid is allowed to merge in a stepwise manner in two or more mixer portions. For example, after merging liquid supplied from two introduction paths in a first mixer portion, the mixture discharged from the aforementioned mixer portion and liquid introduced from the remaining introduction path can be merged in a second mixer portion.

Furthermore, a portion of the raw materials can be preliminarily charged into the flow path of the flow reactor (such as the mixer portion), and the remaining raw materials may be respectively supplied from one or a plurality of raw material introduction ports.

There are no particular limitations on the material of the aforementioned flow reactor, and can be suitably selected corresponding to the required performance, such as heat resistance, pressure resistance, solvent resistance or processing ease. Examples of the aforementioned material include stainless steel, titanium, copper, nickel, aluminum, silicon, fluororesins such as Teflon® or perfluoroalkoxy resin (PFA), trifluoroacetoamide (TFAA) and polyether ether ketone resin (PEEK).

In addition, the material may be substantially the same throughout the entire flow path or different materials may be used for the introduction path, mixer portion and reactor portion, respectively.

There are no particular limitations on the cross-sectional shape of the flow path and may be rectangular, including a square or oblong shape, polygonal, including a triangular or pentagonal shape, star-shaped, or circular, including semicircular or elliptical shape. The cross-sectional shape of the flow path is not required to be constant. Furthermore, “flow path cross-section” refers to the cross-section in a direction perpendicular to the direction of flow of a reaction solution and the like through the flow path, while “cross-sectional area” refers to the area of the aforementioned cross-section.

There are no particular limitations on the cross-sectional area or flow path length of the flow path, and are suitably adjusted in consideration of such factors as viscosity and flow rate of the reaction solution, reaction temperature or reaction time. If the cross-sectional area of the flow path is excessively small, pressure loss becomes high thereby making it difficult to supply raw materials and allow the reaction solution to flow. Conversely, if the cross-sectional area is excessively large, heat exchange efficiency decreases and a temperature distribution and the like occur, thereby reducing the characteristics of the flow reactor. The cross-sectional area of the flow path may be substantially the same throughout the entire flow path or the cross-sectional area may be different in the introduction path, mixer portion and reactor portion, respectively. In the case the aforementioned flow reactor has a plurality of introduction paths, the cross-sectional area of each introduction path may be mutually the same or different.

The mixer portion has a function that mixes a plurality of liquids by dispersion and a function that removes the heat of reaction.

There are no particular limitations on the type of mixing used to mix liquids in the mixer portion, and can be suitably selected corresponding to the particular purpose. For example, mixing may be carried out by laminar flow or turbulent flow.

There are no particular limitations on the mixer portion provided it is provided with a structure that enables mixing of a plurality of liquids, and can be suitably selected corresponding to the particular purpose. Examples of the mixer portion include a T-pipe, micromixer and branched pipe. A T-shape or a Y-shape can be used for the shape of the mixer portion in the case the number of introduction ports is two, while a cross-shape, for example, can be used in the case the number of introduction ports is three.

There are no particular limitations on the cross-sectional area of the mixer portion provided it does not impair the effects of the present invention, and can be suitably adjusted in consideration of such factors such as the type of mixing. Since the mixer portion is able to favorably demonstrate both of the functions of mixing a plurality of liquids by dispersion and removing the heat of reaction, in the case the cross-sectional shape is circular, the mixer portion preferably has an inner diameter of about 10 μm to about 5 cm. In addition, although the cross-sectional area of the mixer portion may be the same as other portions such as the introduction paths, it is preferably larger than the introduction paths from the viewpoint of mixing efficiency.

There are no particular limitations on the flow path length of the mixer portion, and can be suitably adjusted in consideration of such factors as the type of mixing, type and amount of liquid supplied from each introduction path, or the presence or absence of a reactor portion. For example, in the case the cross-sectional shape is circular, the inner diameter can be about 10 μm to about 5 cm and the flow path length can be 10 cm to 50 m.

Although the flow path length of the mixer portion is preferably of a sufficient length for mixing liquids introduced from a plurality of introduction paths by dispersion, in the case of providing a separate reactor portion, the flow path length may be shorter. On the other hand, in the case of not providing a separate reactor portion and obtaining a product following completion of the reaction at the point the liquids have passed through the mixer portion, the flow path length of the mixer portion is preferably suitably adjusted in consideration of the optimum reaction time.

The reactor portion is a site for regulating the length of the flow path and precisely controlling the time required to carry out the reaction (controlling residence time). In a flow reactor, reaction time is equivalent to the residence time in a flow path of a reaction solution obtained by mixing all raw materials. Since the aforementioned residence time is proportional to flow path length, reaction time is adjusted by adjusting flow path length.

The composition such as the cross-sectional area, inner diameter, outer diameter, flow path length and materials of the flow path of the reactor portion can be suitably selected corresponding to the desired reaction. For example, there are no particular limitations on the material of the reactor portion, and those materials listed as examples of materials of the aforementioned flow reactor can be used preferably.

The mixer portion, introduction paths and reactor portion are provided with connecting means for mutually connecting each member as necessary. There are no particular limitations on the connection method used by the aforementioned connecting means, and can be suitably selected from among known tube connection methods corresponding to the particular purpose, with examples of connection methods including threading, union jointing, butt welding, slip-on welding, socket welding, flange connection, flareless connection, flared connection and mechanical connection.

There are no particular limitations on the members other than the introduction ports, mixer portion and reactor portion, and can be suitably selected corresponding to the particular purpose. Examples of the aforementioned members include pumps used to deliver liquid, temperature control means, reaction accelerating means, sensors, pressure regulating valves, and tanks for storing compounds produced.

There are no particular limitations on the aforementioned pumps, and can be suitably selected from among those able to be used industrially. Among these, pumps that do not generate pulsation during delivery of liquid are preferable, and examples thereof include plunger pumps, gear pumps, rotary pumps and diaphragm pumps.

There are no particular limitations on the aforementioned temperature control means, and can be suitably selected corresponding to the reaction temperature. Examples thereof include constant temperature baths, circulators and heat exchangers.

[Sodium Azide and Substituent Y Introducing Agent Reaction Conditions]

There are no particular limitations on the reaction solvent of the reaction solution, which at least contains both an azide and a substituent Y introducing agent and in which an azide compound synthesis reaction occurs in a flow reactor, provided it dissolves the azide and the substituent Y introducing agent and is inert in the aforementioned reaction. Examples of the aforementioned solvent include water, hydrocarbon-based solvents such as pentane, hexane, heptane, benzene, toluene or xylene, nitrile-based solvents such as acetonitrile or propionitrile, ether-based solvents such as diethyl ether, dioxane or tetrahydrofuran, amide-based solvents such as N,N-dimethylformamide, N,N-dimethylacetoamide or N-methylpyrrolidone (NMP), sulfoxide-based solvents such as dimethylsulfoxide and mixed solvents thereof.

In the method for producing an azide compound of the present invention, water or a mixed solvent of water and an organic solvent is preferable due to the favorable solubility of azide therein, and a mixed solvent of water and an organic solvent capable of dissolving an azide compound represented by general formula (I) is more preferable. In the case of recovering the azide compound represented by general formula (I) by separating from unreacted azide represented by general formula (II), a mixed solvent of water and an organic solvent capable of dissolving the azide compound represented by general formula (I) and capable of undergoing phase separation with water is preferably used for the reaction solvent. In this case, the azide compound obtained as a result of synthesis is mainly contained in the organic solvent layer, while the unreacted azide represented by general formula (II) along with salts and other by-products are mainly contained in the aqueous layer. Consequently, the azide compound represented by general formula (I) can be efficiently recovered by recovering the organic solvent layer from the reaction solution following completion of the reaction by separating from the aqueous layer. Hydrocarbon-based solvents are preferable, aromatic hydrocarbon-based solvents are more preferable, toluene, benzene or xylene is even more preferable, and toluene is particularly preferable for the organic solvent capable of dissolving the azide compound represented by general formula (I).

In addition, in the method for producing an azide compound of the present invention, the reaction is preferably carried out in the presence of base. The presence of base in the reaction solution makes it possible to inhibit the generation of hydrogen azide from the azide. Examples of bases include inorganic bases such as sodium hydroxide, potassium hydroxide, sodium hydride, sodium carbonate or potassium carbonate, and organic bases such as triethylamine, 4-(dimethylamino)pyridine, pyridine, 1,8-diazabicyclo[5.4.0]undecene-7 or 1,5-diazabicyclo[4.3.0]nonene-5. One type of these bases can be used alone or two or more types can be used in combination. Among these, alkaline metal hydroxides such as sodium hydroxide or potassium hydroxide are used preferably.

In addition, in the case of using a mixed solvent of water and an organic solvent for the reaction solvent, the reaction solution may further contain a phase transfer catalyst. A phase transfer catalyst refers to a small amount of reagent used to react an organic compound that is insoluble in water with a reagent that is insoluble in organic solvent. There are no particular limitations on the phase transfer catalyst provided it is inert in the reaction between the azide and substituent Y introducing agent, and can be suitably selected from among known phase transfer catalysts in consideration of such factors as the types and combinations of organic solvents used in the reaction solution. Examples of phase transfer catalysts include quaternary ammonium salts in the manner of tetraalkylammonium chlorides such as tetramethylammonium chloride, tetraethylammonium chloride, tetrapropylammonium chloride or tetrabutylammonium chloride (TBAC), tetraalkylammonium bromides such as tetramethylammonium bromide, tetraethylammonium bromide, tetrapropylammonium bromide or tetrabutylammonium bromide, and benzyltrialkylammonium halides such as benzyltrimethylammonium chloride, benzyltrimethylammonium bromide, benzyltri-n-butylammonium chloride (BTBAC) or benzyltri-n-butylammonium bromide. BTBAC is preferably used as a phase transfer catalyst in the method for producing an azide compound of the present invention.

Although there are no particular limitations on the content ratio of azide and substituent Y introducing agent in the reaction solution, which at least contains both the azide and substituent Y introducing agent and in which an azide compound synthesis reaction occurs in a flow reactor, provided it is a ratio that allows the target reaction to proceed, an adequate amount of substituent Y introducing agent relative to the azide is preferably contained in the aforementioned reaction solution.

In the method for producing an azide compound of the present invention, a reaction solution may be prepared in which all raw materials, including the azide and substituent Y introducing agent, have been mixed in advance, the reaction solution is supplied to the flow reactor and allowed to react in a reactor portion of the aforementioned flow reactor to synthesize an azide compound, or the reaction solution may be prepared by respectively supplying a solution containing the azide and a solution containing the substituent Y introducing from separate raw material introduction ports and merging in a mixer portion. From the viewpoint of safety, a method consisting of separately introducing a solution containing the azide and a solution containing the substituent Y introducing agent into the flow reactor is preferable.

The solution containing the azide and the solution containing the substituent Y introducing agent respectively and independently supplied to the flow reactor may be solutions prepared using the same type of solvent or solutions prepared using different types of solvent. For example, the solvent of the solution containing the azide is preferably water or a mixed solvent of water and an organic solvent that does not react with the azide, and is more preferably water. On the other hand, although the solvent of the solution containing the substituent Y introducing agent may be any of water, an organic solvent or a mixed solvent of water and organic solvent, an organic solvent that is inert in the reaction is preferable. In addition, in the case of carrying out the reaction in the presence of base, the base is preferably added to the solution containing the azide. In particular, an aqueous solution containing the azide and base and a solution of the substituent Y introducing agent diluted with organic solvent are preferably separately supplied and merged in the mixer portion. In the case of using a phase transfer catalyst in the aforementioned aspect, the phase transfer catalyst may be added to either the aqueous solution containing the azide or the solution containing the substituent Y introducing agent. In addition, the reaction solution may be prepared by preliminarily housing a solution containing a base in the mixer portion, respectively and separately supplying the aqueous azide solution and the solution of the substituent Y introducing agent diluted with organic solvent from raw material introduction ports, and merging in the mixer portion.

Although there are no particular limitations on the reaction temperature of the reaction solution in the flow reactor (solution following mixing of all raw materials) provided it is a temperature at which risk of decomposition of the synthesized azide compound is adequately inhibited, from the viewpoint of safety, the reaction temperature is preferably a comparatively low temperature. Consequently, the reaction temperature in the method for producing an azide compound of the present invention is preferably 20° C. to 200° C. and more preferably 35° C. to 80° C.

In the method for producing an azide compound of the present invention, an azide compound represented by the aforementioned general formula (I) is obtained in a flow reactor. The resulting azide compound may be washed with neutral to alkaline water following the reaction in order to remove trace amounts of hydrogen azide or sodium azide present in the solution after the reaction.

In the method for producing an azide compound of the present invention, an azide compound represented by the aforementioned general formula (I) is obtained in a flow reactor. In the case of using the resulting azide compound as a raw material of another chemical reaction, that reaction can be successively carried out without recovering the azide compound from the aforementioned flow reactor. Namely, the method for producing an azide compound of the present invention and a subsequent reaction using the azide compound as raw material can be carried out in a single flow reactor (having at least one raw material introduction port and at least one product discharge port as a whole, that are connected by a flow path that is continuous therewith). Since pressure in the flow reactor is easily adjusted, the synthesized azide compound can be supplied to the next reaction step in a liquid state even in the case of having a low boiling point. Carrying out successive reactions in a single flow reactor without having to discharge the synthesized azide compound to the outside makes it possible to carry out a reaction using an azide compound more safely.

<Method for Producing 1H-Tetrazole Derivative>

The method for producing a 1H-tetrazole derivative of the present invention is a method for producing a 1H-tetrazole derivative by synthesizing an azide compound from an azide and substituent Y introducing agent in a single flow reactor followed by reacting with a cyanide compound. Since the reaction is carried out successively in a single flow reactor having a communicating flow path without discharging the intermediate product in the form of the azide compound to the outside, the target 1H-tetrazole derivative can be produced industrially safely.

More specifically, the method for producing a 1H-tetrazole derivative of the present invention is characterized by reacting the aforementioned substituent Y introducing agent and an azide represented by general formula (II) in a solution in a first reactor of a flow reactor to produce an azide compound represented by the aforementioned general formula (I), followed by reacting the aforementioned solution containing the azide compound and a solution containing a cyanide compound represented by the following general formula (IV) by mixing in a second mixer portion of the aforementioned flow reactor to produce a compound represented by the following general formula (V).

[Chemical Formula 11]

N≡CZᵣR¹ₚR²)_{q} (IV)

(In general formula (IV), Z represents —CO—, —SO₂— or —CRₐR_{b}— (wherein, Rₐ and R_{b} respectively and independently represent a hydrogen atom, alkyl group or unsubstituted or substituted aryl group), p represents 0 or 1, q represents 0 or 1, r represents 0 or 1, R¹ represents an alkyl group or hydrogen atom in the case q is 0 or an alkylene group in the case q is 1 and R² represents an unsubstituted or substituted aryl group, provided that q is 1 in the case p is 0 and q is 1 in the case r is 0.)

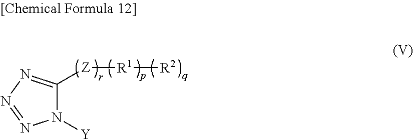

(In general formula (V), Y, Z, R¹, R², p, q and r are the same as previously defined.)

In the method for producing a 1H-tetrazole derivative of the present invention, the step through synthesis of the azide compound is the same as in the aforementioned method for producing an azide compound of the present invention. Next, the following reaction is carried out by allowing the cyanide compound to merge with the resulting azide compound.

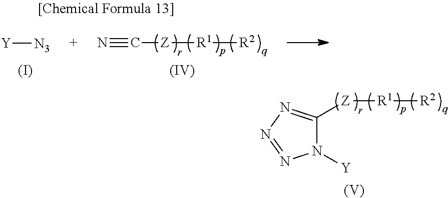

In the method for producing a 1H-tetrazole derivative of the present invention, the azido group of the azide compound represented by general formula (I) is cycloadded to the cyanido group of the cyanide compound represented by general formula (IV). As a result, in comparison with a synthesis method in which a substituent is introduced into a compound having a tetrazole backbone, a 1H-tetrazole derivative having substituents introduced at the 1-position and 5-position can be produced both selectively and efficiently. In addition, by reacting the azide compound and cyanide compound in a flow reactor, the target 1H-tetrazole derivative can be produced efficiently and in a short period of time.

[Cyanide Compound Represented by General Formula (IV)]

In general formula (IV), Z represents —CO—, —SO₂— or —CRₐR_{b}—, p represents 0 or 1, q represents 0 or 1, r represents 0 or 1, R¹ represents an alkyl group or hydrogen atom in the case q is 0 or an alkylene group in the case q is 1, and R² represents an unsubstituted or substituted aryl group, provided that q is 1 in the case p is 0 and q is 1 in the case r is 0. Rₐ and R_{b} respectively and independently represent a hydrogen atom, alkyl group or unsubstituted or substituted aryl group.

In general formula (IV), Z represents —CO— (carbonyl group), —SO₂— (sulfonyl group) or —CRₐR_{b}— (wherein, Rₐ and R_{b} respectively and independently represent a hydrogen atom, alkyl group or unsubstituted or substituted aryl group), and r represents 0 or 1. However, q is always 1 in the case r is 0. In the case Rₐ or R_{b} in Z is an alkyl group, examples of the aforementioned alkyl group are the same as the alkyl groups listed as examples of Y in the aforementioned general formula (I). In addition, in the case Rₐ or R_{b} in Z is an unsubstituted or substituted aryl group, examples of the aforementioned aryl group are the same as the unsubstituted or substituted aryl groups listed as examples of Y in the aforementioned general formula (I).

In the case Z in general formula (IV) represents —CRₐR_{b}—, specific examples thereof include a methylene group (—CH₂—) in which both Rₐ and R_{b} are hydrogen atoms, a group in which either one of Rₐ and R_{b} is a hydrogen atom while the other is an alkyl group, a group in which either one of Rₐ and R_{b} is a hydrogen atom and the other is an unsubstituted or substituted aryl group, and a group in which Rₐ and R_{b} mutually and independently represent an alkyl group or unsubstituted or substituted aryl group. Specific examples of Z in general formula (IV) include the divalent groups indicated below.

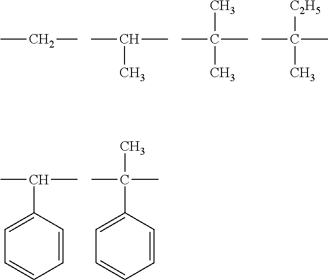

Z in general formula (IV) is more preferably —CO— or —SO₂— than —CRₐR_{b}—. In comparison with using an alkyl nitrile or benzyl cyanide derivative in which a cyano group is bound directly to an alkyl group or alkylaryl group as raw material, the use of a cyanide derivative, in which a cyano group is bound to alkyl group and the like through a carbonyl group or sulfonyl group, allows the reaction to proceed more efficiently.

In addition, a cyanide derivative in which the group adjacent to the cyano group is a carbonyl group tends to result in lower reactivity than a cyanide derivative in which the group adjacent to the cyano group is a sulfonyl group. In the method for producing a 1H-tetrazole derivative of the present invention, since the reaction is carried out in a flow reactor, even in the case of using as a raw material a cyanide derivative in which the group adjacent to the cyano group is a carbonyl group, a 1H-tetrazole derivative can be produced with sufficiently high reaction efficiency and in a short period of time.

In the case q is 0 in general formula (IV), R¹ represents an alkyl group or hydrogen atom. Examples of the aforementioned alkyl group are the same as the alkyl groups listed as examples of Y in the aforementioned general formula (I). R¹ in general formula (IV) preferably represents a linear alkyl group having 1 to 8 carbon atoms, a branched alkyl group having 3 to 8 carbon atoms or a cyclic alkyl group having 3 to 8 carbon atoms.

In the case q is 1 in general formula (IV), R¹ represents an alkylene group. The aforementioned alkylene group may be a linear alkylene group, branched alkylene group or cyclic alkylene group. The aforementioned alkylene group is preferably a linear alkylene group having 1 to 8 carbon atoms, a branched alkylene group having 3 to 8 carbon atoms or a cyclic alkylene group having 3 to 8 carbon atoms, and is more preferably a linear alkylene group having 1 to 6 carbon atoms, a branched alkylene group having 3 to 6 carbon atoms or a cyclic alkylene group having 3 to 6 carbon atoms. Specific examples include a methylene group, ethylene group, n-propylene group, i-propylene group, n-butylene group, i-butylene group, n-pentylene group, n-hexylene group, cyclopropylene group, cyclobutylene group, cyclopentylene group and cyclohexylene group. Among these, R¹ in general formula (IV) in the case q is 1 preferably represents a linear alkylene group having 1 to 3 carbon atoms or a branched alkylene group having 3 carbon atoms, more preferably represents a linear alkylene group having 1 to 3 carbon atoms, and even more preferably represents a methylene group.

The cyanide compound represented by general formula (IV) is preferably [—(R¹)p-(R²)q] from the viewpoint that reaction yield is enhanced as a result of being a group that has highly electron attracting characteristics. Consequently, among compounds represented by general formula (IV), in compounds in which r is 0 or compounds in which r is 1, compounds in which p is 0 or 1 and q is 1 are preferable to compounds in which p is 1 and q is 0, while compounds in which p is 0 and q is 1 are more preferable.

In general formula (IV), R² represents an unsubstituted or substituted aryl group and q represents 0 or 1. However, q is 1 in the case p is 0. The aforementioned aryl group may be a monocyclic group or polycyclic group. Furthermore, as long as at least one ring of a polycyclic aryl group is an aromatic ring, the remaining rings may be saturated rings, unsaturated rings or aromatic rings. In the case R² in general formula (IV) is a substituted aryl group, there are no particular limitations on the aforementioned substituent provided it is a chemically acceptable substituent, and specific examples thereof include the substituents listed in (1) to (85) to be subsequently described.

R² in general formula (IV) preferably represents an aryl group having 6 to 10 carbon atoms and more preferably represents a phenyl group. More specifically, R² is particularly preferably a group represented by the following general formula (s1).

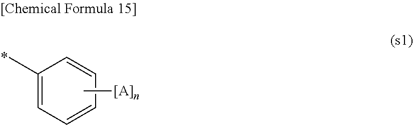

(In general formula (s1), A represents a halogen atom, alkyl group, haloalkyl group, alkoxy group, haloalkoxy group, alkylsulfonyl group, unsubstituted or substituted aryl group, cyano group or nitro group, n represents an integer of 0 to 5, A may be mutually the same or different when n is 2 or more, and the asterisk (*) represents bonding to R¹ in the case p is 1 in general formula (IV), bonding to Z in the p is 0 and r is 1, or binding to a carbon atom of the cyanido group in the case p and r are 0.)

In general formula (s1), n is an integer of 0 to 5, preferably an integer of 0 to 3 and more preferably 0. Furthermore, A may be mutually the same or different when n is 2 or more.

In general formula (s1), A represents a halogen atom, alkyl group, haloalkyl group, alkoxy group, haloalkoxy group, alkylsulfonyl group, unsubstituted or substituted aryl group, cyano group or nitro group.

Examples of halogen atoms include a fluorine atom, chlorine atom, bromine atom and iodine atom.

Examples of alkyl groups include a methyl group, ethyl group, n-propyl group, i-propyl group, n-butyl group, i-butyl group, s-butyl group, t-butyl group, n-pentyl group and n-hexyl group. The number of carbon atoms that compose the alkyl group is preferably 1 to 8.

Examples of haloalkyl groups include a fluoromethyl group, chloromethyl group, bromomethyl group, difluoromethyl group, dichloromethyl group, trifluoromethyl group, trichloromethyl group, trifluoroethyl group, pentafluoroethyl group, 3,3,3,2,2-pentafluoropropyl group and 2,2,2-trifluoro-1-trifluoromethylethyl group. The number of carbon atoms that compose the haloalkyl group is preferably 1 to 8.

Examples of alkoxy groups include a methoxy group, ethoxy group, n-propoxy group, i-propoxy group, n-butoxy group, i-butoxy group, s-butoxy group, t-butoxy group and n-hexyloxy group. The number of carbon atoms that compose the alkoxy group is preferably 1 to 8.

Examples of haloalkoxy groups include a 2-chloro-n-propoxy group, 2,3-dichlorobutoxy group and trifluoromethoxy group. The number of carbon atoms that compose the haloalkoxy group is preferably 1 to 8.

Examples of alkylsulfonyl groups include a methylsulfonyl group, ethylsulfonyl group, n-propylsulfonyl group, i-propylsulfonyl group and t-butylsulfonyl group. The number of carbon atoms that compose the alkylsulfonyl group is preferably 1 to 8.

An aryl group refers to a monocyclic or polycyclic aryl group. Furthermore, as long as at least one ring of a polycyclic aryl group is an aromatic ring, the remaining rings may be saturated rings, unsaturated rings or aromatic rings. Among aryl groups, aryl groups having 6 to 10 carbon atoms are preferable.

Specific examples of unsubstituted aryl groups include a phenyl group, 1-napthyl group, 2-naphthyl group, azulenyl group, indanyl group and tetralinyl group.

There are no particular limitations on “substituents” in a substituted aryl group provided they are chemically acceptable substituents. Specific examples thereof include the following substituents:

(1) halogen atoms such as a fluorine atom, chlorine atom, bromine atom or iodine atom; (2) alkyl groups such as a methyl group, ethyl group, n-propyl group, i-propyl group, n-butyl group, s-butyl group, i-butyl group, t-butyl group, n-pentyl group or n-hexyl group; (3) cycloalkyl groups such as a cyclopropyl group, cyclobutyl group, cyclopentyl group, cyclohexyl group or cycloheptyl group; (4) alkoxy groups such as a methoxy group, ethoxy group, n-propoxy group, i-propoxy group, n-butoxy group, i-butoxy group, s-butoxy group or t-butoxy group; (5) alkenyl groups such as a vinyl group, 1-propenyl group, 2-propenyl group, 1-butenyl group, 2-butenyl group, 3-butenyl group, 1-methyl-2-propenyl group, 2-methyl-2-propenyl group, 1-pentenyl group, 2-pentenyl group, 3-pentenyl group, 4-pentenyl group, 1-methyl-2-butenyl group, 2-methyl-2-butenyl group, 1-hexenyl group, 2-hexenyl group, 3-hexenyl group, 4-hexenyl group or 5-hexenyl group;

(6) cycloalkenyl groups such as a 2-cyclopropenyl group, 2-cyclopentenyl group, 3-cyclohexenyl group or 4-cyclooctenyl group; (7) alkenyloxy groups such as a vinyloxy group, allyloxy group, 1-propenyloxy group or 2-butenyloxy group; (8) alkynyl groups such as an ethynyl group, 1-propynyl group, 2-propynyl group, 1-butynyl group, 2-butynyl group, 3-butynyl group, 1-methyl-2-propynyl group, 2-methyl-3-butynyl group, 1-pentynyl group, 2-pentynyl group, 3-pentynyl group, 4-pentynyl group, 1-methyl-2-butynyl group, 2-methyl-3-pentynyl group, 1-hexynyl group or 1,1-dimethyl-2-butynyl group; (9) alkynyloxy groups such as an ethynyloxy group or propargyloxy group; (10) aryl groups such as a phenyl group, 1-naphthyl group or 2-naphthyl group;

(11) aryloxy groups such as a phenoxy group or 1-naphthoxy group; (12) aralkyl groups such as a benzyl group or phenethyl group; (13) aralkyloxy groups such as a benzyloxy group or phenethyloxy group; (14) acyl groups such as a formyl group, acetyl group, propionyl group, benzoyl group, cyclohexylcarbonyl group or phthaloyl group; (15) alkoxycarbonyl groups such as a methoxycarbonyl group, ethoxycarbonyl group, n-propoxycarbonyl group, i-propoxycarbonyl group, n-butoxycarbonyl group or t-butoxycarbonyl group; (16) carboxyl groups; (17) hydroxyl groups; (18) haloalkyl groups such as a chloromethyl group, chloroethyl group, 1,2-dichloro-n-propyl group, 1-fluoro-n-butyl group or perfluoro-n-pentyl group; (19) haloalkoxy groups such as a 2-chloro-n-propoxy group, 2,3-dichlorobutoxy group or trifluoromethoxy group; (20) haloalkenyl groups such as a 2-chloro-1-propenyl group or 2-fluorol-butenyl group; (21) haloalkynyl groups such as a 4,4-dichloro-1-butynyl group, 4-fluoro-1-pentynyl group or 5-bromo-2-pentynyl group;

(22) haloalkenyloxy groups such as a 2-chloro-1-propenyloxy group or 3-bromo-2-butenyloxy group; (23) haloalkynyl groups such as a 3-chloropropargyl group or 3-iodopropargyl group; (24) haloalkynloxy groups such as a 3-chloropropargyloxy group or 3-iodopropargyloxy group; (25) haloaryl groups such as a 4-chlorophenyl group, 4-fluorophenyl group or 2,4-dichlorophenyl group; (26) haloaryloxy groups such as a 4-fluorophenoxy group or 4-chloro-1-naphthoxy group; (27) halogen-substituted acyl groups such as a chloroacetyl group, trifluoroacetyl group, trichloroacetyl group or 4-chlorobenzoyl group; (28) alkoxyalkyl groups such as a methoxymethyl group, ethoxymethyl group, 1-ethoxyethyl group or 2-ethoxyethyl group; (29) alkoxyalkoxy groups such as a methoxymethoxy group, ethoxymethoxy group, 1-ethoxyethoxy group or 2-ethoxyethoxy group; (30) cyano groups;

(31) isocyano groups; (32) nitro groups; (33) isocyanato groups; (34) cyanato groups; (35) amino groups (NH₂ groups); (36) alkylamino groups such as a methylamino group, dimethylamino group or diethylamino group; (37) arylamino groups such as an anilino group, naphthylamino group or anthranylamino group; (38) aralkylamino groups such as a benzylamino group or phenethylamino group; (39) alkylsulfonylamino groups such as a methylsulfonylamino group, ethylsulfonylamino group, n-propylsulfonylamino group, i-propylsulfonylamino group or n-butylsulfonylamino group; (40) arylsulfonylamino groups such as a phenylsulfonylamino group;

(41) heteroarylsulfonylamino groups such as a pyrazinylsulfonylamino group; (42) acylamino groups such as a formylamino group, acetylamino group, propanoylamino group, butyrylamino group, i-propylcarbonylamino group or benzoylamino group; (43) alkoxycarbonylamino groups such as a methoxycarbonylamino group or ethoxycarbonylamino group; (44) haloalkylsulfonylamino groups such as a fluoromethylsulfonylamino group, chloromethylsulfonylamino group, bromomethylsulfonylamino group, difluoromethylsulfonylamino group, dichloromethylsulfonylamino group, 1,1-difluoroethylsulfonylamino group, trifluoromethylsulfonylamino group, 2,2,2-trifluoroethylsulfonylamino group or pentafluoroethylsulfonylamino group; (45) bis(alkylsulfonyl)amino groups such as bis(methylsulfonyl)amino group, bis(ethylsulfonyl)amino group, (ethylsulfonyl)(methylsulfonyl)amino group, bis(n-propylsulfonyl)amino group, bis(i-propylsulfonyl)amino group, bis(n-butylsulfonyl)amino group or bis(t-butylsulfonyl)amino group;

(46) bis(haloalkylsulfonyl)amino groups such as a bis(fluoromethylsulfonyl)amino group, bis(chloromethylsulfonyl)amino group, bis(bromomethylsulfonyl)amino group, bis(dichloromethylsulfonyl)amino group, bis(1,1-difluoroethylsulfonyl)amino group, bis(trifluoromethylsulfonyl)amino group, bis(2,2,2-trifluoroethylsulfonyl)amino group or bis(pentafluoroethylsulfonyl)amino group; (47) unsubstituted or substituted hydrazino groups such as a hydrazino group, N′-phenylhydrazino group, N′-methoxycarbonylhydrazino group, N′-acetylhydrazino group or N′-methylhydrazino group; (48) unsubstituted or substituted aminocarbonyl groups such as a aminocarbonyl group, dimethylaminocarbonyl group, phenylaminocarbonyl group or N-phenyl-N-methyaminocarbonyl group; (49) unsubstituted or substituted hydrazinocarbonyl groups such as a hydrazinocarbonyl group, N′-methylhydrazinocarbonyl group or N′-phenylhydrazinocarbonyl group; (50) unsubstituted or substituted iminoalkyl groups such as a N-methyliminomethyl group, 1-N-phenyliminoethyl group, N-hydroxyiminomethyl group or N-methoxyiminomethyl group;

(51) thiol groups; (52) isothiocyanato groups; (53) thiocyanato groups; (54) alkylthio groups such as a methylthio group, ethylthio group, n-propylthio group, i-propylthio group, n-butylthio group, i-butylthio group, s-butylthio group or t-butylthio group; (55) alkynylthio groups such as a vinylthio group or allylthio group; (56) alkynylthio groups such as a ethynylthio group or propargylthio group; (57) arylthio groups such as a phenylthio group or napthylthio group; (58) heteroarylthio groups such as a 2-pyridylthio group or 3-pyridazylthio group; (59) aralkylthio groups such as a benzylthio group or phenethylthio group; (60) heteroarylalkylthio groups such as a 2-pyridylmethylthio group or 2-furylmethylthio group; (61) alkylthiocarbonyl groups such as a methylthiocarbonyl group, ethylthiocarbonyl group, n-propylthiocarbonyl group, i-propylthiocarbonyl group, n-butylthiocarbonyl group, i-butylthiocarbonyl group, s-butylthiocarbonyl group or t-butylthiocarbonyl group;

(62) alkylthioalkyl groups such as a methylthiomethyl group or 1-methylthioethyl group; (63) arylthioalkyl groups such as phenylthiomethyl group or 1-phenylthioethyl group; (64) alkylthioalkoxy groups such as a methylthiomethoxy group or 1-methylthioethoxy group; (65) arylthioalkoxy groups such as a phenylthiomethoxy group or 1-phenylthioethoxy group; (66) alkylsulfinyl groups such as a methylsulfinyl group, ethylsulfinyl group or t-butylsulfinyl group; (67) alkenylsulfinyl groups such as an allylsulfinyl group; (68) alkynylsulfinyl groups such as a propargylsulfinyl group; (69) arylsulfinyl groups such as a phenylsulfinyl group; (70) heteroarylsulfinyl groups such as a 2-pyridylsulfinyl group or 3-pyridylsulfinyl group; (71) aralkylsulfinyl groups such as a benzylsulfinyl group or phenethylsulfinyl group; (72) heteroarylalkylsulfinyl groups such as a 2-pyridylmethylsulfinyl group or 3-pyridylmethylsulfinyl group;

(73) alkylsulfonyl groups such as a methylsulfonyl group, ethylsulfonyl group or t-butylsulfonyl group; (74) alkenylsulfonyl groups such as an allylsulfonyl group; (75) alkynylsulfonyl groups such as a propargylsulfonyl group; (76) arylsulfonyl groups such as a phenylsulfonyl group; (77) heteroarylsulfonyl groups such as a 2-pyridylsulfonyl group or 3-pyridylsulfonyl group; (78) aralkylsulfonyl groups such as a benzylsulfonyl group or phenethylsulfonyl group; (79) heteroarylalkylsulfonyl groups such as a 2-pyridylmethylsulfonyl group or 3-pyridylmethylsulfonyl group; (80) unsaturated 5-membered heterocyclic groups such as a furan-2-yl group, furan-3-yl group, thiophen-2-yl group, thiophen-3-yl group, pyrrol-2-yl group, pyrrol-3-yl group, oxazol-2-yl group, oxazol-4-yl group, oxazol-5-yl group, thiazol-2-yl group, thiazol-4-yl group, thiazol-5-yl group, isoxazol-3-yl group, isoxazol-4-yl group, isoxazol-5-yl group, isothiazol-3-yl group, isothioazol-4-yl group, isothiazol-5-yl group, imidazol-2-yl group, imidazol-4-yl group, imidazol-5-yl group, pyrazol-3-yl group, pyrazol-4-yl group, pyrazol-5-yl group, 1,3,4-oxadiazol-2-yl group, 1,3,4-thiadiazol-2-yl group, 1,2,3-triazol-4-yl group, 1,2,4-triazol-3-yl group or 1,2,4-triazol-5-yl group;

(81) unsaturated 6-membered heterocyclic groups such as a pyridin-2-yl group, pyridin-3-yl group, pyridine-4-yl group, 5-chloro-3-pyridyl group, 3-trifluoromethyl-2-pyridyl group, pyridazin-3-yl group, pyridazin-4-yl group, pyrazin-2-yl group, pyrimidin-5-yl group, 1,3,5-triazin-2-yl group or 1,2,4-triazin-3-yl group; (82) saturated or partially unsaturated heterocyclic groups such as a tetrahydrofuran-2-yl group, tetrahydropyran-4-yl group, piperidin-3-yl group, pyrrolidin-2-yl group, morpholino group, piperidino group, N-methylpiperazino group or oxazolin-2-yl group; (83) heterocyclooxy groups such as a 2-pyridyloxy group or 3-isoxazolyloxy group; (84) heteroarylalkyl groups such as a 2-pyridylmethyl group or 3-pyridylmethyl group; and, (85) heteroarylalkoxy groups such as a 2-pyridylmethoxy group or 3-pyridylmethoxy group.

These substituents exemplified in (1) to (85) are able to further have the substituents exemplified in (1) to (85) therein within a chemically acceptable range.

Specific examples of substituted aryl groups include a 4-fluorophenyl group, 4-chlorophenyl group, 2,4-dichlorophenyl group, 3,4-dichlorophenyl group, 3,5-dichlorophenyl group, 2,6-difluorophenyl group, 4-trifluoromethylphenyl group, 4-methoxyphenyl group, 3,4-dimethoxyphenyl group, 3,4-methylenedioxyphenyl group, 4-trifluoromethoxyphenyl group, 4-methoxy-1-naphthyl group, 4-ethoxyphenyl group and 4-methylphenyl group.

Among these, the compound represented by general formula (s1) is preferably that in which n is an integer of 0 to 3 and A represents a halogen atom, alkyl group or alkoxy group, more preferably that in which n is an integer of 0 to 3 and A represents a halogen atom, and even more preferably a compound in which n is 0.

The cyanide compound represented by general formula (IV) is preferably a compound in which r is 0 or 1, p is 0 or 1, R¹ represents a linear alkylene group having 1 to 3 carbon atoms or a branched alkylene group having 3 carbon atoms, q is 1 and R² is a group represented by general formula (s1), more preferably a compound represented by the following general formulas (IV-1) to (IV-48), even more preferably a compound represented by the following general formulas (IV-1) to (IV-32), still more preferably a compound represented by the following general formula (IV-1), (IV-9), (IV-17) or (IV-25), and particularly preferably a compound represented by the following general formula (IV-1) or (IV-9). In general formulas (IV-1) to (IV-48), R^{1′} represents a linear alkylene group having 1 to 3 carbon atoms or a branched alkylene group having 3 carbon atoms and X represents a halogen atom.

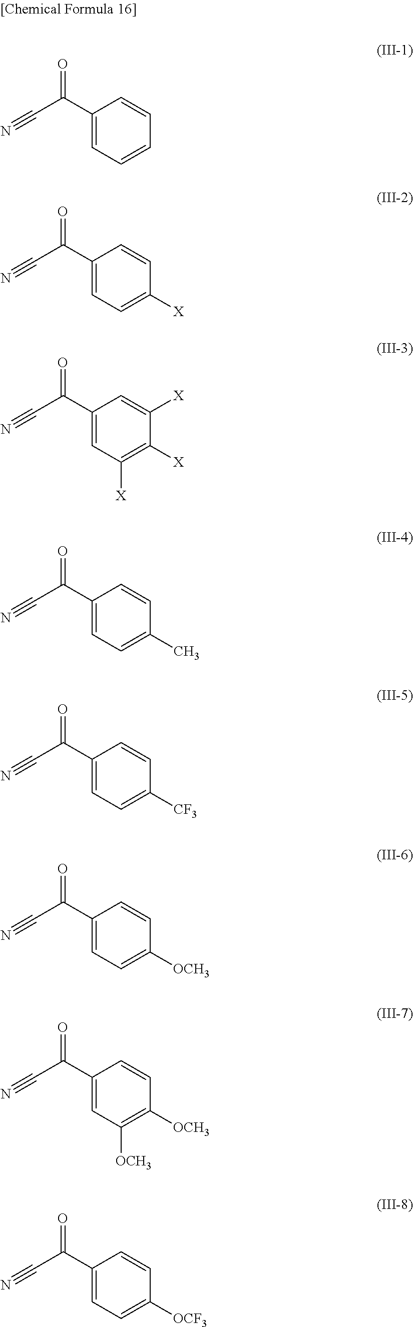

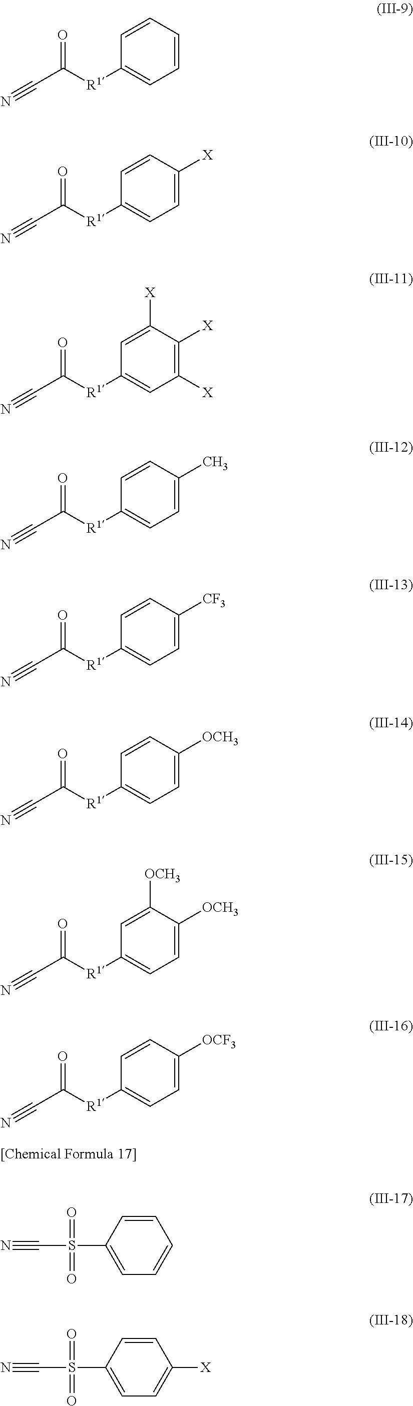

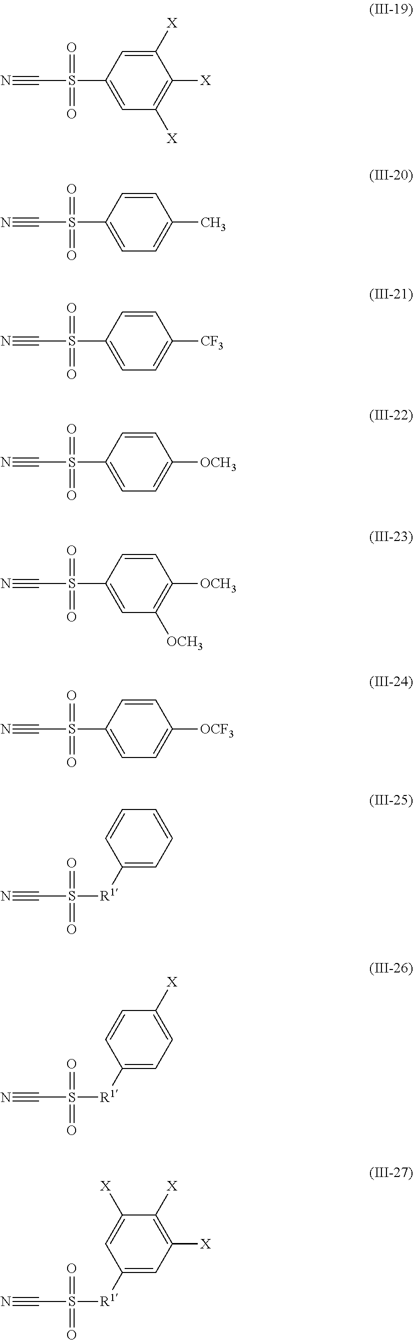

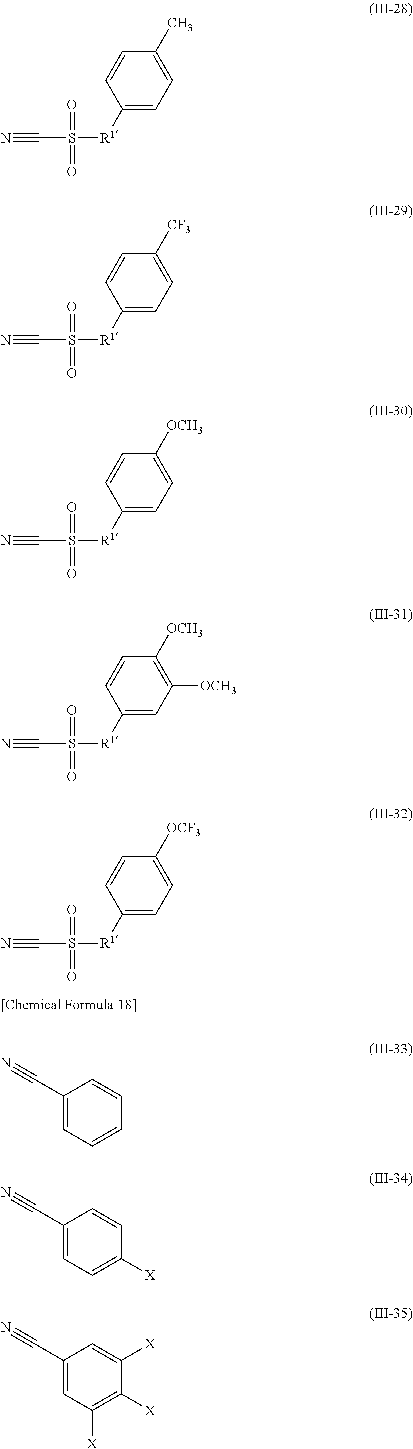

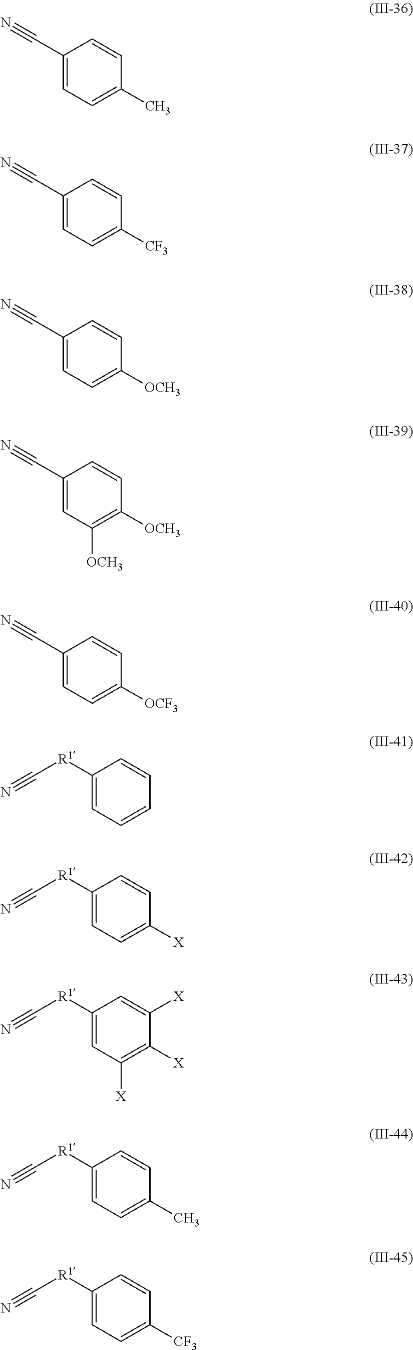

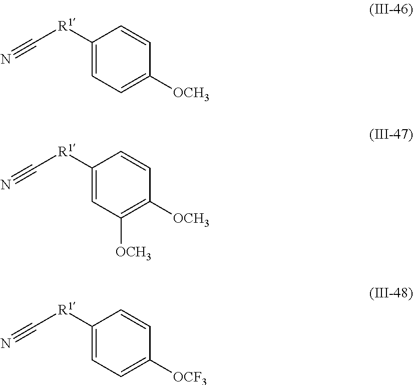

The cyanide compound represented by general formula (IV) can be synthesized from a known compound using a known chemical reaction. For example, among cyanide compounds represented by general formula (IV), a compound (benzoylcyanide derivative) in which Z represents a carbonyl group, p is 0, q is 1, r is 0 or 1 and R² is a group represented by general formula (s1) can be produced by reacting a benzoyl halide derivative and a cyanide derivative.

[Azide Compound and Cyanide Compound Reaction Conditions]

Among the reactions in the flow reactor, there are no particular limitations on the solvent of the reaction solution when the azide compound represented by general formula (I) (to also be simply referred to as the “azide compound”) is reacted with the cyanide compound represented by general formula (IV) (to also be simply referred to as the “cyanide compound”) provided it dissolves both the cyanide compound and the azide compound, is inert in the cycloaddition reaction of the azide compound to the cyanido group of the cyanide compound, and has a boiling point sufficiently higher than the reaction temperature. Examples of the aforementioned solvent include hydrocarbon-based solvents such as pentane, hexane, heptane, benzene, toluene or xylene, nitrile-based solvents such as acetonitrile or propionitrile, ether-based solvents such as diethyl ether, dioxane or tetrahydrofuran, amide-based solvents such as N,N-dimethylformamide, N,N-dimethylacetoamide or N-methylpyrrolidone (NMP), sulfoxide-based solvents such as dimethylsulfoxide, water, and mixed solvents thereof. In addition, an acid such as acetic acid may be added to these organic solvents. In the method for producing a 1H-tetrazole derivative of the present invention, a hydrocarbon-based solvent or amide-based solvent is used preferably, while toluene or NMP is used more preferably.

Although there are no particular limitations on the content ratio of the cyanide compound and azide compound in the reaction solution when reacting the cyanide compound and azide compound in the flow reactor provided it allows the target reaction to proceed, an adequate amount of azide compound relative to the cyanide compound is preferably contained in the reaction solution. For example, the content of the azide compound is preferably 1.4 times or more, more preferably 1.8 times or more, even more preferably 2 times or more, still more preferably 3 times or more, and particularly preferably 4 times or more, the number of moles of the cyanide compound.

There are no particular limitations on the reaction temperature when reacting the cyanide compound and the azide compound provided it is a temperature that adequately suppresses the risk of decomposition of the azide compound. For example, the reaction is preferably carried out at 150° C. to 250° C. and more preferably carried out at 150° C. to 220° C.

[Flow Reactor]

The flow reactor used in the method for producing a 1H-tetrazole derivative of the present invention is the flow reactor used in the method for producing an azide compound of the present invention that has a mixer portion for merging a solution containing the cyanide compound downstream from the reactor portion where the reaction for synthesizing the azide compound is carried out (and in the case a reactor portion is omitted, a mixer portion where a solution containing the azide and a solution containing the substituent Y introducing agent are merged), and has a reactor portion as necessary.

More specifically, a flow reactor can be used that is provided with a first mixer portion, introduction paths respectively communicating with the aforementioned first mixer portion and at least two raw material introduction ports, a first reactor portion downstream from the aforementioned first mixer portion, a flow path communicating with the aforementioned first reactor portion and a second mixer portion located downstream therefrom, introduction paths respectively communicating with the aforementioned second mixer portion and at least one raw material introduction port, and a flow path communicating with the aforementioned second mixer portion and a product discharge port. A flow reactor from which the first mixer portion has been omitted can be used in the case of introducing preliminarily prepared solutions containing the azide and the substituent Y introducing agent into the flow reactor. In addition, a reactor portion may also be provided downstream from the second mixer portion (but upstream from the product discharge portion) as necessary.

In the case a solution containing the substituent Y introducing agent and a solution containing the azide are respectively and separately introduced into the flow reactor, the reaction is specifically carried out in the manner indicated below. First, the solution containing the azide and the solution containing the substituent Y introducing agent are respectively supplied from separate raw material introduction ports and merged in the first mixer portion followed by introducing the resulting mixed liquid into the first reactor portion. Within the aforementioned first reactor portion (and in the aforementioned first mixer portion depending on the case), both compounds react and an azide compound is synthesized. Furthermore, in the case the synthesis reaction proceeds sufficiently in the first mixer portion only, the first mixer portion also functions as the first reactor portion and the first reactor portion need not be provided separate from the first mixer portion. A solution containing the azide compound discharged from the first reactor portion (or from the first mixer portion that also functions as the first reactor portion) is introduced into the second mixer portion and is merged with the solution containing the cyanide compound supplied from a separate raw material introduction port to synthesize a 1H-tetrazole derivative. A solution containing the 1H-tetrazole derivative discharged from the second mixer portion (or from a reactor portion located downstream therefrom) is recovered from the product discharge port.

A liquid separation portion for separating into an aqueous layer and an organic solvent layer may be provided between the first reactor portion (or the first mixer portion in the case of also functioning as the first reactor portion) and the second mixer portion. For example, in the case the reaction solvent of the azide compound synthesis reaction was a mixed solvent of water and an organic solvent, the solution containing the synthesized azide compound is separated into an aqueous layer and an organic solvent layer prior to being introduced into the second mixer portion, and the aforementioned organic solvent layer is recovered and introduced into the second mixer portion where it is merged with the solution containing the cyanide compound.

### EXAMPLES

Although the following provides a more detailed explanation of the present invention through examples thereof, the present invention is not limited to these examples.

### Example 1

Methyl azide was synthesized by reacting sodium azide and dimethyl sulfate in a flow reactor.

<Flow Reactor>

An SUS pipe (2 mm (inner diameter)×2.8 m (flow path length), residence time during flow rate of 1 mL/min: approximately 9 min) equipped with a back pressure valve and having one end connected to a T-mixer (material: PEEK) and the other end connected to a condenser was immersed in a water bath at 40° C. and used as a flow reactor. 50 mL volume glass syringes (syringe A, syringe B) were respectively connected to the remaining two openings of the T-mixer not connected to the SUS pipe, and these glass syringes were respectively attached to syringe pumps.

<Solution Preparation>

19.56 g (0.30 mol) of sodium azide, 25.77 g of 28% by weight aqueous sodium hydroxide solution (final concentration: 0.72 mol/L) and 9.42 g of 50% by weight aqueous BTBAC solution (final concentration: 0.06 mol/L) were dissolved with 214.4 g of water to prepare a sodium azide solution. The sodium azide concentration of the resulting sodium azide solution was 7.27% by weight (1.2 mol/L).

Separate from the above, 56.83 g of dimethyl sulfate were diluted with 53.72 g of toluene to prepare a dimethyl sulfate solution. The dimethyl sulfate concentration of the resulting dimethyl sulfate solution was 53.17% by weight (4.5 mol/L).

<Reaction>

35.32 g of the sodium azide solution were filled into syringe A and 24.35 g of the dimethyl sulfate solution were filled into syringe B. Next, the solutions were delivered from each syringe so that the ratio between the liquid volume delivered from syringe A and the liquid volume delivered from syringe B (volume ratio) was 7:3 and the total flow rate was 1 ml/min. As a result, 34.51 g of the sodium azide solution from syringe A (charged amount of sodium azide: 38.46 mmol) and 14.2 g of the dimethyl sulfate solution from syringe B (charged amount of dimethyl sulfate: 59.7 mmol, equal to 1.55 times the number of moles of sodium azide) were introduced into the SUS pipe. Subsequently, a reaction solution obtained by cooling with the condenser was recovered.

The recovered reaction solution was separated into an aqueous layer and an organic solvent layer (toluene layer) and respective amounts of methyl azide and sodium azide contained therein were analyzed by HPLC. The yields (mol %) of methyl azide and sodium azide in each layer based on the charged amount of sodium azide are shown in Table 1. As a result, methyl azide was contained in both the organic solvent layer and aqueous layer and methyl azide was able to be synthesized safely under low temperature conditions of 40° C. without requiring a special explosion-proof apparatus. In addition, although the entire amount of unreacted sodium azide was contained in the aqueous layer, nearly 90% of the synthesis product in the form of methyl azide had migrated to the organic solvent layer.

TABLE 1
Yield (mol %)

| | MeN₃ | NaN₃ |
|---|---|---|
| Organic solvent layer | 38.3 | 0.0 |
| Aqueous layer | 5.6 | 28.9 |
| Total | 43.9 | 28.9 |

### Example 2

Methyl azide was synthesized by reacting sodium azide and dimethyl sulfate in a flow reactor.

<Flow Reactor>

An SUS pipe (1 mm (inner diameter)×19.2 m (flow path length), residence time during flow rate of 1.6 mL/min: approximately 9 min) equipped with a back pressure valve and having one end connected to a T-mixer (material: PEEK) and the other end connected to a condenser was immersed in an oil bath at 80° C. and used as a flow reactor. HPLC pumps A and B were respectively connected to the remaining two openings of the T-mixer not connected to the reactor portion with an SUS pipe.

<Solution Preparation>

3.9 g (0.06 mol) of sodium azide and 10.29 g of 28% by weight aqueous sodium hydroxide solution (equivalent to 120 mol % based on sodium azide) were weighed into a 50 mL volumetric flask and brought to a final volume of 50 mL with water to prepare a sodium azide solution. The sodium azide concentration of the resulting sodium azide solution was 1.2 mol/L.

Separate from the above, 30.27 g (equivalent to 400 mol %) of dimethyl sulfate were weighed into a 100 mL volumetric flask and brought to a final volume of 100 mL with toluene to prepare a dimethyl sulfate solution. The dimethyl sulfate concentration of the resulting dimethyl sulfate solution was 2.4 mol/L and half that amount was delivered.

<Reaction>

The sodium azide solution was placed in the aspiration port of the HPLC pump A and the dimethyl sulfate solution was placed in the aspiration port of the HPLC pump B. The HPLC pump A and the HPLC pump B were operated for 60 minutes so that the respective delivery volume was 0.8 mL/min (total flow rate: 1.6 mL/min). As a result, 50 mL of sodium azide solution (charged amount of sodium azide: 0.06 mol) from HPLC pump A and 50 mL of dimethyl sulfate solution (charged amount of dimethyl sulfate: 0.12 mol, equal to 2.0 times the number of moles of sodium azide) from HPLC pump B were introduced into the SUS pipe. Subsequently, pump A was switched to water and pump B was switched to toluene as washing liquids followed by operating for 60 minutes and recovering the reaction solution after cooling with the condenser.

The recovered reaction solution was separated into an aqueous layer and an organic solvent layer (toluene layer) and respective amounts of methyl azide and sodium azide contained therein were analyzed by HPLC. The yields (mol %) of methyl azide and sodium azide in each layer based on the charged amount of sodium azide are shown in Table 2. As a result, methyl azide was contained in both the organic solvent layer and aqueous layer. Yield improved to 74.8 mol % and methyl azide was able to be synthesized safely and at high yield even under temperature conditions of 80° C. without requiring a special explosion-proof apparatus. In addition, although the entire amount of unreacted sodium azide was contained in the aqueous layer, nearly 80% of the synthesis product in the form of methyl azide had migrated to the organic solvent layer.

TABLE 2
Yield (mol %)

| | MeN₃ | NaN₃ |
|---|---|---|
| Organic solvent layer | 57.20 | 0.00 |
| Aqueous layer | 17.57 | 10.43 |
| Total | 74.77 | 10.43 |

### INDUSTRIAL APPLICABILITY

The present invention is able to provide a method for efficiently and safely producing an azide compound in a flow reactor by using an azide of an alkaline metal or alkaline earth metal as raw material.

According to the method for producing an azide compound and the method for producing a 1H-tetrazole derivative of the present invention, since an azide compound and a 1H-tetrazole derivative that has substituents at the 1-position and 5-position, which are useful as synthesis raw materials of tetrazoyl oxime derivatives that are useful as active ingredients of agricultural chemicals, pharmaceuticals and the like, can be produced efficiently and safely, the method for producing an azide compound and the method for producing a 1H-tetrazole derivative of the present invention can be used in manufacturing fields such as agricultural chemicals or pharmaceuticals.

1. A method for producing an azide compound, comprising: reacting an alkylating agent or silylating agent with an azide represented by the following general formula (II):
[Chemical Formula 1]
M(N₃)ₘ (II)
(wherein, M represents an alkaline metal atom or alkaline earth metal atom and m represents 1 or 2) in a flow reactor to produce an azide compound represented by the following general formula (I):
[Chemical Formula 2]
Y—N₃ (I)
(wherein, Y represents an alkyl group, arylalkyl group, substituted silyl group or substituted silylalkyl group) in the state of a solution.

2. The method for producing an azide compound according to claim 1, wherein the alkylating agent or the silylating agent is a compound represented by any of the following general formulas (III-A) to (III-E): 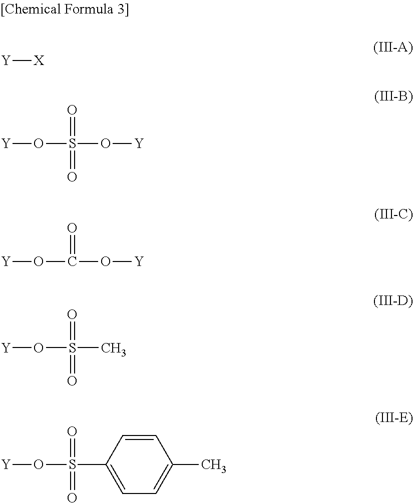
(wherein, Y is the same as defined in the general formula (I) and X represents a halogen atom).

3. The method for producing an azide compound according to claim 1, wherein a solution containing the alkylating agent or the silylating agent and a solution containing the azide represented by the general formula (II) are respectively and separately introduced into the flow reactor.

4. The method for producing an azide compound according to claim 3, wherein the solvent of the solution containing the azide represented by the general formula (II) contains water, and the solvent of the solution containing the alkylating agent or the silylating agent is an organic solvent capable of dissolving the azide compound represented by the general formula (I) and capable of undergoing phase separation with water.

5. The method for producing an azide compound according to claim 4, wherein the organic solvent is toluene.

6. The method for producing an azide compound according to claim 3, wherein the solution containing the azide represented by the general formula (II) further contains a base.

7. The method for producing an azide compound according to claim 1, wherein Y represents an alkyl group.

8. A method for producing a 1H-tetrazole derivative, comprising: reacting an alkylating agent or silylating agent with an azide represented by the following general formula (II):
[Chemical Formula 4]
M(N₃)ₘ (II)
(wherein, M represents an alkaline metal atom or alkaline earth metal atom and m represents 1 or 2) in a solution in a first reactor portion of a flow reactor to produce an azide compound represented by the following general formula (I):
[Chemical Formula 5]
Y—N₃ (I)
(wherein, Y represents an alkyl group, arylalkyl group, substituted silyl group or substituted silylalkyl group), followed by mixing the solution containing the azide compound and a solution containing a cyanide compound represented by the following general formula (IV):
[Chemical Formula 6]
N≡CZᵣR¹ₚR²)_{q} (IV)
(wherein, Z represents —CO—, —SO₂— or —CRₐR_{b}— (wherein, Rₐ and R_{b} respectively and independently represent a hydrogen atom, alkyl group or unsubstituted or substituted aryl group), p represents 0 or 1, q represents 0 or 1, r represents 0 or 1, R¹ represents an alkyl group or hydrogen atom in the case q is 0 or an alkylene group in the case q is 1 and R² represents an unsubstituted or substituted aryl group, provided that q is 1 in the case p is 0 and q is 1 in the case r is 0) in a second mixer portion of the flow reactor and allowing to react to produce a compound represented by the following general formula (V): 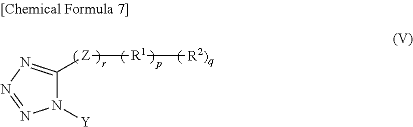
(wherein, Y, Z, R¹, R², p, q and r are the same as previously defined).

9. The method for producing a 1H-tetrazole derivative according to claim 8, wherein the alkylating agent or the silylating agent is a compound represented by any of the general formulas (III-A) to (III-E): 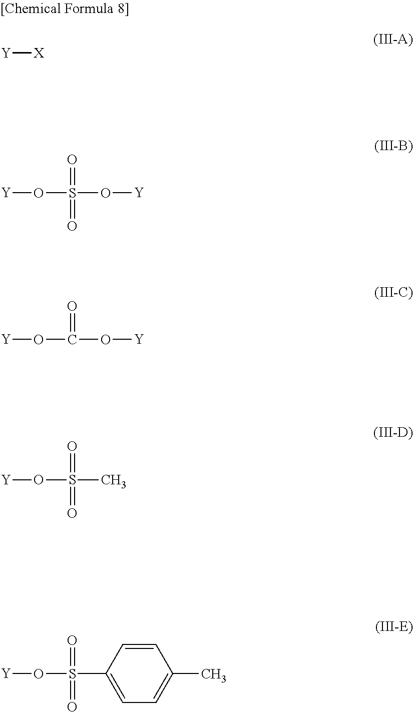
(wherein, Y is the same as defined in the general formula (I) and X represents a halogen atom).

10. The method for producing an azide compound according to claim 8, wherein a solution containing the alkylating agent or the silylating agent and a solution containing the azide represented by the general formula (II) are respectively and separately introduced into the flow reactor, both solutions are mixed in the first mixer portion, and the resulting mixture is introduced into the first reactor portion.

11. The method for producing a 1H-tetrazole derivative according to claim 10, wherein the solvent of the solution containing the azide represented by the general formula (II) contains water, and the solvent of the solution containing the alkylating agent or the silylating agent is an organic solvent capable of dissolving the azide compound represented by the general formula (I) and capable of undergoing phase separation with water.

12. The method for producing a 1H-tetrazole derivative according to claim 11, wherein the organic solvent is toluene.

13. The method for producing a 1H-tetrazole derivative according to claim 11, wherein the solution containing the azide compound represented by the general formula (I) discharged from the first reactor portion is separated into an aqueous layer and an organic solvent layer, and the organic solvent layer is recovered and mixed with a solution containing the cyanide compound represented by the general formula (IV) in the second mixer portion.

14. The method for producing an azide compound according to claim 10, wherein the solution containing the azide represented by the general formula (II) further contains a base.

15. The method for producing a 1H-tetrazole derivative according to claim 8, wherein Y represents an alkyl group.

16. The method for producing a 1H-tetrazole derivative according to claim 8, wherein R² is a group represented by the following general formula (s1): 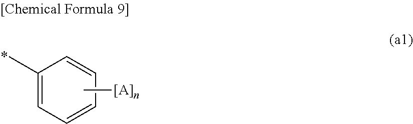
(wherein, A represents a halogen atom, alkyl group, haloalkyl group, alkoxy group, haloalkoxy group, alkylsulfonyl group, unsubstituted or substituted aryl group, cyano group or nitro group, n represents an integer of 0 to 5, A may be the same or different when n is 2 or more, and the asterisk (*) represents bonding to R¹ in the case p is 1 in the general formula (IV), bonding to Z in the case p is 0 and r is 1, or binding to a carbon atom of a cyanido group in the case p and r are 0).

17. The method for producing a 1H-tetrazole derivative according to claim 8, wherein the reaction temperature of the reaction between the azide compound represented by the general formula (I) and the cyanide compound represented by the general formula (IV) is 150° C. to 250° C.

18. The method for producing a 1H-tetrazole derivative according to claim 8, wherein the solution containing the azide compound represented by the general formula (I) that is mixed with the solution containing the cyanide compound represented by the general formula (IV) contains the azide compound represented by the general formula (I) in an amount equal to 1.4 times or more the number of moles of the cyanide compound represented by general formula (IV).

19. The method for producing a 1H-tetrazole derivative according to claim 8, wherein the solvent of the solutions mixed in the second mixer portion is toluene or N-methylpyrrolidone.
